(19) United States
(12) Patent Application Publication
(10) Pub. No.: US 2024/0139145 A1
(43) Pub. Date: May 2, 2024

(54) NON-AQUEOUS INJECTABLE COMPOSITION OR SUSTAINED RELEASE OF BUPRENORPHINE AND USE THEREOF

(71) Applicant: NAVIN SAXENA RESEARCH AND TECHNOLOGY PRIVATE LIMITED, Mumbai (IN)

(72) Inventors: Kunal SAXENA, Mumbai (IN); Navin SAXENA, Mumbai (IN)

(21) Appl. No.: 18/547,044

(22) PCT Filed: Feb. 18, 2022
(86) PCT No.: PCT/IN22/50133

§ 371 Date: Aug. 18, 2023

(30) Foreign Application Priority Data
Feb. 18, 2021 (IN) 202121006827

(51) Int. Cl. A61K 31/33; A61K 9/00; A61K 47/10; A61K 47/14; A61K 47/24; A61K 47/34

(52) U.S. Cl. CPC A61K 31/33; A61K 9/0019; A61K 47/10; A61K 47/14; A61K 47/24; A61K 47/34

(57) ABSTRACT

The present invention discloses a sustained release non-aqueous injectable composition of buprenorphine, its metabolite, its prodrug, or its salt thereof for the treatment of moderate-to-severe pain and opioid dependence. The non-aqueous injectable composition includes one or more biocompatible non-aqueous solvents, one or more release retarding agents, one or more surfactants, one or more biodegradable polymers and one or more stabilizers. The buprenorphine composition of the present invention delivers buprenorphine over a period of about one week to 12 weeks after single administration which leads to better patient compliance by reducing the daily dose administration of buprenorphine. The present invention also provides an injectable composition with potentially less pain due to the use of smaller gauge needles wherein the suitable gauge of needle may be 23 gauge to 30 gauge with a length of 8 to 30 mm. The preferred size of needle is 25 to 30 gauge.

### FIELD OF THE INVENTION

The present invention relates to sustained release injectable formulation of buprenorphine and its metabolites, its prodrug, or its salts thereof for long term delivery of buprenorphine for about 7 days to 3 months as a patient compliant treatment option for chronic pain and opioid de-addiction. The sustained release injectable formulation of buprenorphine of the present invention is a low viscosity solution, ensuring content uniformity and injectability.

### BACKGROUND OF THE INVENTION

Buprenorphine (also known as (2S)-2-[(−)-(5R,6R,7R,14S)-9α-cyclo-propyl-methyl-4,5-epoxy-6,14-ethano-3-hydroxy-6-methoxymorphinan-7-yl]-3,3-di-methylbutan-2-ol.

The chemical structure of buprenorphine is shown in formula (1):

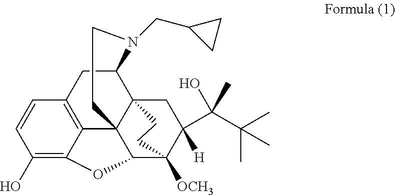 [SMILES: CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

Buprenorphine is a partial opioid agonist. This means that, like opioids, it produces effects such as analgesia, euphoria or respiratory depression. With buprenorphine, however, these effects are weaker than those of full drugs such as heroin and methadone. Buprenorphine's opioid effects increase with each dose until at moderate doses they level off, even with further dose increases. This “ceiling effect” lowers the risk of misuse, dependency, and side effects. Therefore, it is primarily used to treat opioid addiction, acute pain, and chronic pain.

Continuous research works have been carried out to formulate suitable treatment option for opioid addiction, acute pain, and chronic pain. Still there remain some un-met needs related to the need for administering buprenorphine on a daily basis and going to de-addiction centre for administering the medicine.

The buprenorphine hydrochloride injection for intravenous or intramuscular administration was approved under the brand name Buprenex with the dosage of 0.3 mg/ml for alleviating pain. This product can deliver the therapeutic levels of buprenorphine up to several hours. The patients are required to take this medication at regular intervals multiple times daily. Purdue Pharma L.P. has launched a transdermal patch of buprenorphine for maintaining the relief of moderate to severe pain and chronic pain for 7 days that releases buprenorphine at the time intervals of 5, 10 and 20 μg/hr and during the course of the treatment about 15% of the drug is utilized within the body.

The buprenorphine is also administered as sublingual tablets and buccal films to treat the symptoms arising from opioid addiction and for the long term relief of pain. Currently, the commercial products, Subutex® (buprenorphine) sublingual tablet and Suboxone® (buprenorphine and naloxone) sublingual tablets are used for the treatment of opioid dependence and are marketed by RB Pharma Inc. These tablet formulations are intended to deliver therapeutic levels of buprenorphine for short periods of time of up to several hours and are typically taken either buccally or sublingually. However, the patient is required to supplement with the dose of buprenorphine at regular intervals, and there are often issues with diversion in patients with an opioid dependence problem.

These sublingual tablets are also used for de-addiction programs. The formulations containing buprenorphine can be misused because of its opioid effects, particularly by people who do not have an opioid dependency. So, these compositions are not given as take home medicine to the patients and the patients need to visit the de-addiction centre daily for administering the medicine. Thus there is a need therefore for a long term, non-divertible method of administering buprenorphine which delivers a constant and effective dose of the active to the patient over a sustained period of time.

However, buprenorphine-naloxone formulations have been developed as take home medicament for de-addiction. Naloxone is added to buprenorphine to decrease the likelihood of diversion and misuse of the combination drug product. A buccal film product based on a combination of buprenorphine and naloxone is available in the US to treat opioid dependence. When these products are taken as sublingual tablets, buprenorphine's opioid effects will be dominated. If the sublingual tablets are crushed and injected, however, the naloxone effect dominates and may cause opioid withdrawals.

To alleviate the need of taking medication daily, once stabilized with the drug, the patients are treated further by using sustained release injections or implants. Probuphine® is a buprenorphine implant, which delivers the drug up to 6 months. Four Probuphine® implants are inserted subdermally in the upper arm for 6 months of treatment and are removed by the end of the sixth month. The insertion & removal of implant is a tedious procedure, which reduces the patient compliance. To increase the patient compliance, the biodegradable microsphere formulations are most commonly used in the injectable sustained release formulations of buprenorphine. Due to the manufacturing complexity of microsphere formulations, more emphasis is made on in-situ gelling composition, so as to maintain uniformity and ease of administration. An injectable oily solution containing 6 mg/ml to 10 mg/ml buprenorphine base discloses a sustained release of buprenorphine in mice for 51 hours to 75 hours (Liu et. al., 2006). An in vitro release of 30-50 days using in-situ gelling technology with Buprenorphine hydrochloride:PLGA ratio (1:50) was reported (Grave et. al., 2007). A solution of 15 mg/ml buprenorphine base in different non-aqueous solvents like triethylcitrate, acetyl triethyl citrate, tributyl citrate, acetyl tributyl citrate, benzyl benzoate, ethyl benzoate provided 15-60% in vitro release of buprenorphine in 5 days (Xu, 2012).

The US patents assigned to Indivior UK limited U.S. Pat. Nos. 10,022,367 and 10,517,864 which discloses the non-polymeric compositions containing 8% w/w to 50% w/w buprenorphine in N-methyl pyrrolidone (NMP)/Dimethyl acetamide/Dimethyl suphoxide; U.S. Pat. Nos. 9,498,432 and 9,827,241 discloses 8-30% buprenorphine in 15-70% solution of Poly (D, L-lactide-co glycolide) in NMP; U.S. Pat. No. 9,295,645 discloses 10-20% buprenorphine base in aqueous suspension, where these injectable formulations show a sustained release of buprenorphine for a period of a week or a month. In November 2018, one such formulation of Indivior, “Sublocade™” has been approved by USFDA as a first once-monthly injectable buprenorphine formulation for the treatment of moderate to severe opioid use disorder (OUD) in patients. Sublocade™ is based on biodegradable Atrigel polymer delivery technology. The Atrigel polymer delivery technology includes a poly (DL-lactide-co-glycolide) (PLGA) or poly (DL-lactide) (PLA) with N-methyl pyrrolidone (NMP) is disclosed in US Patent Application no. 2016/0128997. The major components in Sublocade™ include a poly (DL-lactide-co-glycolide) (PLGA) with N-methylpyrrolidone (NMP) and the active ingredient, buprenorphine is disclosed in U.S. Pat. No. 8,921,387. Even though Sublocade™ prepared by dissolving buprenorphine in Atrigel system can be filled in a prefilled syringe without mixing step before usage, there are still other drawbacks, for example due to high viscosity of formulation, a large needle (19 gauge) for the injection is required which may cause discomfort in patient and decrease the patient compliance. Also, the safety of the amount of NMP used as a solvent in buprenorphine injection composition is yet to be established. Besides, the colour change over time (from colourless initially to amber) of buprenorphine composition may be another indicator for the stability issue.

A PCT application WO2020077235 discloses the method for treating or preventing fentanyl-induced respiratory depression and opioid use disorder by administering a composition of buprenorphine in an amount of about 18 wt %, along with a poly (lactide-co-glycolide) copolymer in an amount of about 32 wt % and N-methyl-2-pyrrolidone in an amount of about 50 wt % wherein the composition is administered once per month by subcutaneous injection.

Another subcutaneously administrated sustained release buprenorphine dosage was approved by the USFDA under trade name of Buvidal®/Brixadi™ in late 2018 which is administered as a once weekly and a once monthly injection for treating moderate to severe opioid use disorder (OUD). Buvidal® encapsulates buprenorphine by a lipid-based matrix with a tradename as Fluid Crystal®. The major components in Buvidal® include a phospholipid, such as phosphatidylcholine, diacyl ester, such as glycerol dioleate, solvent, such as ethanol or NMP, and the active ingredient, buprenorphine is disclosed in patents U.S. Pat. Nos. 8,236,755 and 9,937,164. FluidCrystal® also forms solid depot after injection, in which phosphatidyl choline would gradually lyse adipose tissue and may aggravate the discomfort (Rotunda, A M., Kolodney, M. S. Mesotherapy and Phosphatidylcholine Injections: Historical Clarification and Review. Dermatologic Surgery, (2006) 32(4), 465-480). Moreover, though the volume of single injection (0.64 ml) contributed by Buvidal® seems smaller than the volume of Sublocade™ (1.5 ml), the cumulative injected volume of the composition in a month of Buvidal® may be as large as 2.6 ml. Thus, the discomfort may be not less than the discomfort caused by Sublocade injection.

Both commercial subcutaneous injection buprenorphine product, including Sublocade™ manufactured by Indivior and Buvidal® manufactured by Camurusare not colour stable. Both changes colour over time, from colourless to amber (FDA advisory committee meeting briefing document: Indivior RBP-6000: pp 30 Oct. 31, 2017 and Braeburn Pharmaceutical CAM2038 pp 29 Nov. 1, 2017). Not only for the aesthetic issue, this discoloration would increase the challenge of chemistry, manufacturing and control (CMC) because colour is always a key attribute in specification of parenteral pharmaceuticals. Additionally, based on label information, both products are granted with 18-month shelf-life, which is not ideal from an aspect of small-molecule drug products.

Camurus has worked on a polymeric depot system for controlled release matrix formulation using an injectable solution containing 12% w/w of buprenorphine in at least one oxygen carrying organic solvent as phospholipid, acyl glycerol and N-methyl-2-pyrrolidone that maintains plasma concentration of approximately 10 ng/ml to 60 ng/ml up to 56 days in rats after a single injection of 60 mg/kg (AU2017279657 B and WO2014016428). The patent AU2017279657 B shows that the Cₘₐₓ was above 100 ng/mL after administration of 60 mg/kg (approx. equivalent to 600 mg Human dose) of the phospholipid formulation in rats (with average body weight ˜300 g), which gradually declined to 10 ng/mL in 35 days. The Cₘₐₓ/C_{min.} ratio is approx. 10. The formulation with less C_{max.}/C_{min.} ratio is desirable for sustained maintenance of plasma concentration of buprenorphine.

Novel drug delivery systems like unilamellar liposomes and multi vesicular liposomes can also lead to sustained delivery of the drug. For example DOXIL, PEGylated liposomes of doxorubicin hydrochloride reduces the dose of doxorubicin hydrochloride to once in 3-4 week as compared to daily dose of conventional injection. Depocyt, a multi vesicular liposome of cytarabine is a once in 2-4 week formulation. However, these novel formulations need special raw materials and manufacturing technique, which reduce their affordability.

For better patient compliance, there is a need of developing sustained release drug delivery system of buprenorphine which is affordable, safe and also easy to administer.

The present invention therefore aims to provide such an affordable injection that is cost-effective, painless, as it is low viscous solution and which does not need any special manufacturing equipment or control. The sustained release buprenorphine injection of the present invention may be administered using a fine needle.

### SUMMARY OF THE INVENTION

The present invention provides homogeneous liquid composition which can be injected into a body and which is likely to cause less pain on injection. Unlike implant, there is no need of making an incision into a body for administration. The sustained release buprenorphine composition of the present invention when injected into the body, it forms depot at the site of injection, which gradually releases the drug into the blood stream by diffusion.

According to one aspect, the invention provides a non-aqueous, sustained release injectable composition with a polymer and a release retarding agent.

According to another aspect, the non-aqueous composition of the present invention does not need a complex manufacturing technique or process controls as required in most of the microsphere based sustained release injections. This ensures robust and reproducible manufacturing of the formulation.

According to another aspect, the invention provides a non-aqueous, sustained release injectable composition which is used for a method of treating opioid addiction, acute pain and chronic pain.

According to another aspect, the invention provides a non-aqueous, sustained release injectable composition which is formulated for administration by subcutaneous injection or intramuscular injection.

According to another aspect, the invention provides an injectable composition of buprenorphine wherein after a single administration the formulation will deliver buprenorphine over a period of about one week to 12 weeks, thereby reducing daily dose administration of buprenorphine.

### BRIEF DESCRIPTION OF DRAWINGS

FIG. 1 illustrates the release profile of buprenorphine in the composition which consists of benzyl alcohol mixed with benzyl benzoate. A nearly zero-order release pattern was observed in initial 35 days, with approximately 90% cumulative release by 35 days. It is apparent from the figure that slow drug release of buprenorphine could be achieved in the formulation having benzyl alcohol and benzyl benzoate.

FIG. 2 illustrates the release profile of buprenorphine in the composition which consists of about 30% benzyl alcohol, 20% benzyl benzoate and sesame oil as release retardant. In the release study performed by dialysis method, approximately 93% of drug was released in 27 days.

FIG. 3 illustrates the release profile of buprenorphine in the composition which consists of about 3.5% polycaprolactone as release retardant, benzyl benzoate and 20% benzyl alcohol. The release study performed by dialysis method at 37±0.2° C. shows that a constant release of buprenorphine was observed in the range of 3 mg/day to 5 mg/day.

FIG. 4 illustrates the release profile of buprenorphine in the composition which consists of about 20% buprenorphine, 20% hydrogenated soya phosphatidyl choline (HSPC) and benzyl alcohol. In the release study performed by dialysis method at 37±0.2° C., slow release of buprenorphine was observed with approximately 14% was observed in 22 days.

FIG. 5 illustrates the buprenorphine plasma concentrations in rats after a single subcutaneous injection of different buprenorphine composition at 10 mg/kg dose. It is apparent from the figure that drug release occurred in a controlled manner up to 35 days.

FIG. 6 illustrates the buprenorphine plasma concentrations in rats after a single subcutaneous injection of different buprenorphine composition at 4 mg/kg dose. It is apparent from the figure that the drug release occurred in a controlled manner from all the formulations of example 6 up to 14 days.

### DESCRIPTION OF THE INVENTION

The present invention is directed to a sustained release delivery system for buprenorphine.

Accordingly, the present invention provides a long term, non-aqueous injectable pharmaceutically acceptable composition for sustained release of buprenorphine, its active metabolite, its prodrug or its salt thereof, comprising one or more biocompatible non-aqueous solvents with one or more release retarding agents and its method of use for the treatment of opioid addiction, moderate to severe pain, acute pain and chronic pain, comprising administering an effective amount of the composition of buprenorphine subcutaneously or intramuscularly.

The pharmaceutical composition of the present invention provides enhanced ability of the buprenorphine composition to maintain appropriate properties such as injectability and chemical stability of the composition. The composition according to the present invention delivers a therapeutically effective level of buprenorphine for at least 7 days, preferably at least about 28 days, or at least about 3 months to treat patients for opioid dependence, pain and other indications.

As used herein, the singular forms “a,” “an,” and “the” include plural reference unless the context clearly dictates otherwise. Thus, for example, a reference to “a non-aqueous injectable composition” includes a plurality of such formulations, so that a formulation of buprenorphine includes formulations of buprenorphine.

As used herein, the term “pharmaceutically acceptable” means that the material, substance, compound, molecule, polymer, or system to which it applies should not cause severe toxicity, severe adverse biological reaction, or lethality in an animal to which it is administered at reasonable doses and rates.

As used herein, the term “pharmaceutically acceptable salts” refer to derivatives wherein the parent compound is modified by making acid or base salts thereof. Suitable acceptable salts include, but are not limited to, mineral or organic acid salts of basic residues such as amines; alkali or organic salts of acidic residues such as carboxylic acids; and the like. The acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluene sulfonic, methane-sulfonic, ethane disulfonic, oxalic, isethionic, and the like. Specifically, the acceptable salts can include, for example, those salts that naturally occur in vivo in a mammal.

As used herein, the term “therapeutically effective amount” is intended to include an amount of buprenorphine, a pharmaceutically acceptable salt thereof, useful to treat or prevent the underlying disorder or disease, or to treat the symptoms associated with the underlying disorder or disease in a host.

As used herein, the terms “treating,” “treat,” or “treatment” includes (i) preventing a pathologic condition (e.g., opioid use disorder (OUD)) from occurring (e.g., prophylaxis); (ii) inhibiting the pathologic condition (e.g., OUD) or arresting its development; and (iii) relieving the pathologic condition (e.g., relieving the symptoms associated with OUD).

In one of the embodiments, a pharmaceutically acceptable non-aqueous injectable composition comprises one or more biocompatible non-aqueous solvents as a vehicle with one or more release retarding agents for maintaining uniform release rate of the drug from the injectable composition.

Accordingly, the present invention provides a sustained release injectable pharmaceutical composition, comprising:
a. buprenorphine, its metabolite, a prodrug, or a salt thereof present in the concentration of about 0.5% w/w to 40.0% w/w of total composition;
b. a vehicle at a concentration of about 5 w/w to 95% w/w of total composition;
c. a release retarding agent at a concentration of about 0.2% w/w to 40% w/w of total composition; and
d. a stabilizer at a concentration of about 5 w/w to 70% w/w of total composition.

In another embodiment, the present invention provides a high concentration of a non-viscous solution of buprenorphine which can be easily injected using small gauge needles. The buprenorphine solution provided in the present invention is filled in a prefilled syringe containing buprenorphine solution in the volume of 1 ml, 2.2 ml, 5 ml and 10 ml or ampoule or vials having a fill volume range from 0.1 ml to 4 ml. The said Buprenorphine formulation is presented in a pre-filled syringe it then ensures accuracy and consistency of dose administered, minimal wastage and stability of the product. The injectable composition of buprenorphine of the present invention is less viscous than the other existing injectable formulations of buprenorphine using a polymer, which requires the size of the needles larger than 19 gauge. Due to less viscosity, the composition of present invention can be injected using finer needle (preferably 23-27 gauge), which may lead to less pain on injection.

In another embodiment, the appropriate addition of pharmaceutically acceptable vehicle significantly reduces the viscosity of highly concentrated buprenorphine formulation, thus improving the injectability and reducing the force during injection, including breakthrough and gliding force. The vehicle used in the composition is a non-aqueous solvent or a lipophilic solvent or in combinations thereof present in a range of 5% w/w to 95% w/w of total formulation by weight. In another embodiment, the pharmaceutically acceptable non-aqueous solvent is selected from the group consisting of polyethylene glycol, propylene glycol, benzyl alcohol, benzyl benzoate, isopropyl myristate, ethyloleate either individually or in any mixtures thereof.

In another embodiment, the pharmaceutically acceptable lipophilic solvent is selected from the group consisting of soybean oil, sesame oil, peanut oil, cottonseed oil, castor oil, arachis oil and the like individually or in any mixtures thereof.

In another embodiment, the addition of pharmaceutically acceptable release retarding agent in the composition provides slow, constant and sustained release of drug over a prolong period. The pharmaceutically acceptable release retarding agent used in the composition is selected from the group consisting of a phospholipid or a polymer or a lipophilic chemical entity and the like individually or in any mixtures thereof wherein the releasing retarding agent is present in a range of 0.2% w/w to 40% w/w of total formulation by weight.

In another embodiment, the pharmaceutically acceptable phospholipids are added in the composition. The pharmaceutically acceptable phospholipid used in the composition is selected from the group consisting of a natural, semisynthetic and synthetic phospholipid.

In another embodiment, the pharmaceutically acceptable phospholipid used in the composition is in the form of di-esters which are selected from the group consisting of phosphocholines, glycerol phospholipids, phosphatidylserines and sphingomyelin. The phosphocholine is selected from the group consisting of hydrogenated soya phosphatidyl choline (HSPC), 1,2-distearoyl-sn-glycero-3-phosphocholine (DSPC), dipalmitoyl phosphatidylcholine (DPPC), 1,2-dimyristoyl-sn-glycero-3-phospho-choline (DMPC), 1-palmitoyl-2-oleoyl-snglycero-3-phosphocholine (POPC), soya phosphatidyl choline (SPC) and soyalecithin.

In another embodiment, the pharmaceutically acceptable phospholipid used in the composition is supplemented with additives for release modification, which are selected from the group consisting of cholesterol, Ethyl Hexyl Oleate, glyceryl esters like glyceryl monooleate, glyceryl monostearate, glyceryl palmitate, glyceryl palmitostearate, glyceryl dibehenate, glyceryl distearate, glyceryl isostearate, glyceryl laurate, glyceryl mono and dicaprylocaprate, glyceryl mono and dipalmitostearate, glyceryl monocaprylate, glyceryl monocaprylocaprate, glyceryl monocitrate, glyceryl oleate, glyceryl palmitate, glyceryl ricinoleate, glyceryl stearate, glyceryl stearate/peg-100 stearate, glyceryl tristearate, polyoxyl glyceryl stearate, sucrose esters, glycol esters. In another embodiment, the drug encapsulated in the pharmaceutically acceptable biodegradable polymer which is released over extended period of time and hence eliminates the need for multiple injections. This feature can improve patient compliance especially for drugs like buprenorphine for chronic indications, requiring frequent injection. The pharmaceutically acceptable biodegradable polymer added in the composition is a polyorthoester selected from polycaprolactone (PCL) and polylactic acid (PLA).

In another embodiment, the continuous release of buprenorphine from the polymer matrix could occur either by diffusion of the drug from the polymer matrix or by the erosion of polymer or by combination of two mechanisms.

In another embodiment, the pharmaceutically acceptable lipophilic chemical entity which is added as surfactants into the composition in order to increase drug absorption, bioavailability of drug and slows release of the drug. The pharmaceutically acceptable lipophilic chemical entity is selected from the group consisting of stearyl alcohol, cetostearyl alcohol, cetyl alcohol, stearic acid, palmitic acid, stearates, palmitates, fatty acid and fatty acid esters and wax which is selected from the group consisting of carnauba wax, beeswax and hydrogenated castor oil.

In another embodiment, the pharmaceutically acceptable stabilizer is used in the formulation for parenteral administration of buprenorphine to prevent oxidation and stability issues due to colour change of buprenorphine in the composition. The pharmaceutically acceptable stabilizer is selected from the group consisting of benzyl alcohol, butylated hydroxyl anisole (BHA), butylated hydroxyl toluene (BHT) and alpha-tocopherol and the like individually or in any mixtures thereof in a range of 0.5% w/w to 70% w/w of total formulation by weight.

The sustained release injectable pharmaceutical composition provided according to the invention further includes an additive, which can be selected from the group consisting of cholesterol, glycerylmonooleate, glycerylmonostearate and glyceryl palmitate.

In an embodiment, the sustained release injectable pharmaceutical composition according to the present invention can be administered by subcutaneous injection or intramuscular injection, wherein the pharmaceutical composition after single administration will deliver buprenorphine over a period of about one week to 3 months, thereby reducing daily dose administration of buprenorphine.

In an embodiment of the present invention, the bioavailability of buprenorphine as measured from time zero to time of last measurement for a single dose, as the area under the curve of plasma concentration against time in rats is 275 to 795 ng/ml*hours per mg of buprenorphine.

In yet another embodiment, the invention provides a method of treating opioid addiction, acute pain and chronic pain, comprising administering an effective amount of the composition provided in accordance with the present invention.

### EXAMPLES

### Example 1

### Buprenorphine Base Solution

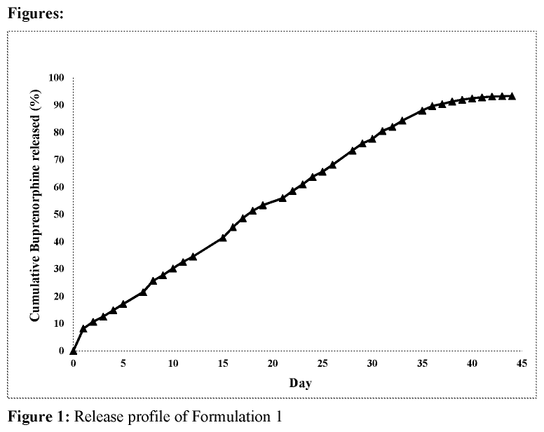

Buprenorphine (7 g) was added to 14 mL benzyl alcohol & stirred for 30 min. Benzyl benzoate was added to this mixture with continuous stirring, sufficient to make up the volume to 70 mL. The solution thus formed was a clear solution. Release study was performed by dialysis method at 37±0.2° C. In FIG. 1, a nearly zero-order release pattern was observed in initial 35 days, with approximately 90% cumulative release in 35 days.

### Example 2

### Composition Containing a Release Retardant in a Suitable Oil

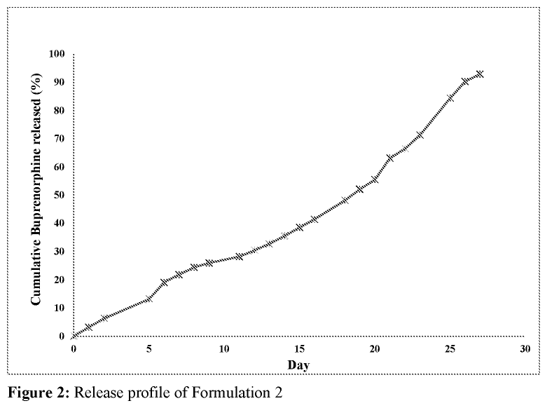

A solution was prepared, wherein 0.5 g buprenorphine was added to 1.4 mL benzyl alcohol followed by the addition of 1.0 mL benzyl benzoate. Sesame oil was further added to this solution to make up the volume to 5 mL. The solution thus formed was a clear solution. In FIG. 2, a release study performed by dialysis method showed that approximately 93% of buprenorphine is released in 27 days. A reduction in burst release was also observed.

### Example 3

### Composition Containing Polycaprolactone as a Release Retardant

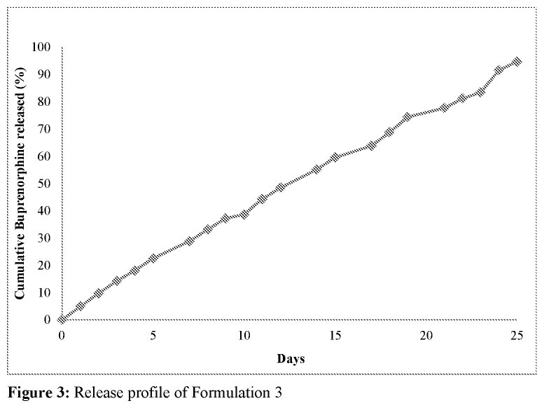

A solution comprising of 10% w/v buprenorphine, 3.5% w/v polycaprolactone and 20% v/v benzyl alcohol was prepared in Benzyl benzoate. This clear viscous solution was subjected to release study at 37±0.2° C. using dialysis method. As shown in FIG. 3, a nearly constant release of buprenorphine was observed, with a release profile of 3 mg/day to 5 mg/day up to 25 days.

### Example 4

### Composition Containing Phospholipid as a Release Retardant

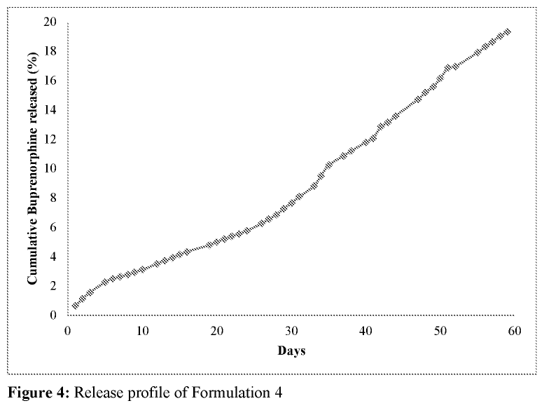

A solution comprising of 110 g buprenorphine and 110 g hydrogenated soya phosphatidyl choline (HSPC) was prepared in benzyl alcohol, sufficient to make up the volume to 550 mL. This clear viscous solution was subjected to release study at 37±0.2° C. using dialysis method. In FIG. 4, a release study showed very slow release of approximately 14% of drug was released in 42 days.

This formulation of example 4 is a stable, clear, colourless to light yellowish solution. When stored below 25° C., the solution remains clear & colourless as shown in table below.

| | | | | | | | 40 ± 2° C./ |
|---|---|---|---|---|---|---|---|
| | | | | | 25 ± 2° C./60 ± 5% | | 75 ± 5% |
| | | | 5 ± 3° C | | RH | | RH |
| Test | | INITIAL | 3 M | 18 M | 3 M | 18 M | 3 M |
| Description | | Clear | Clear | Clear | Clear | Clear | Clear |
| | | colourless | colourless | colourless | colourless | colourless | yellow |
| | | solution | solution | solution | solution | solution | solution |
| Assay | | 93.2 | 99.5 | 94.72 | 99.7 | 92.73 | 99.4 |
| Impurity | Unknown | 0.05 | 0.08 | 0.06 | 0.13 | 0.04 | 0.22 |
| | Total | 0.05 | 0.14 | 0.09 | 0.34 | 0.07 | 0.49 |

### Example 5

In Vivo Pharmacokinetic Study in Rats at 10 mg/kg

For monthly duration, different formulations (A to D) were prepared using different additives & different concentrations, as listed in table 2. Formulation D was prepared similar to marketed formulation Sublocade 100 mg composition for comparison.

| Group | Treatment | Brief composition | | |
|---|---|---|---|---|
| G1 | Formulations A | Buprenorphine | 72.6 | g |
| | | HSPC | 80 | g |
| | | Benzyl alcohol | q.s. 400 mL | |
| G2 | Formulations B | Buprenorphine | 9.07 | g |
| | | HSPC | 10 | g |
| | | Cholesterol | 2.5 | g |
| | | Benzyl alcohol | q.s. 50 mL | |
| G3 | Formulations C | Buprenorphine | 9.07 | g |
| | | HSPC | 10 | g |
| | | Glyceryl monooleate | 1.25 | g |
| | | Benzyl alcohol | q.s. 50 mL | |
| G4 | Formulations D | Buprenorphine | 20.0 | g |
| | | PLGA | 35.6 | g |

| | |
|---|---|
| NMP | 55.6 g (q.s. to 100 mL) |

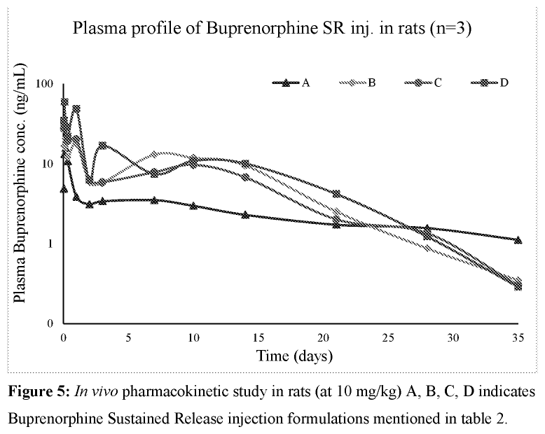

Male Sprague Dawley rats were administered formulations containing 10 mg/kg buprenorphine using #23 gauge needle, subcutaneously. The Sublocade like Formulation (Formulation D) needed #20 gauge needle for injection, because of its viscous nature. Blood samples were withdrawn up to 35 days & plasma concentration was determined using LCMS. The results (FIG. 5) showed that a formulation of buprenorphine prepared with phospholipid resulted into controlled delivery of buprenorphine in vivo which is evident from more uniform plasma concentration of buprenorphine in the compositions with phospholipid, when compared to product similar to marketed formulation (administered to G4). Moreover, a reduction in burst release was also observed in formulations A to C when compared to formulation D.

### Example 6

In Vivo Pharmacokinetic Study in Rats at 4 mg/kg

For weekly to fortnightly duration, different formulations were prepared using different additives & different concentrations, as listed in Table 3.

| Group | Treatment | Brief composition | | |
|---|---|---|---|---|
| G1 | Formulations AW | Buprenorphine | 5.04 | g |
| | | HSPC | 10 | g |
| | | Benzyl alcohol | q.s. 100 mL | |
| G2 | Formulations BW | Buprenorphine | 5.04 | g |
| | | HSPC | 10 | g |
| | | Cholesterol | 3 | g |
| | | Benzyl alcohol | q.s. 100 mL | |
| G3 | Formulations CW | Buprenorphine | 5.04 | g |
| | | HSPC | 10 | g |
| | | Glyceryl monooleate | 2.5 | g |

| | |
|---|---|
| Benzyl alcohol | q.s. 100 mL |

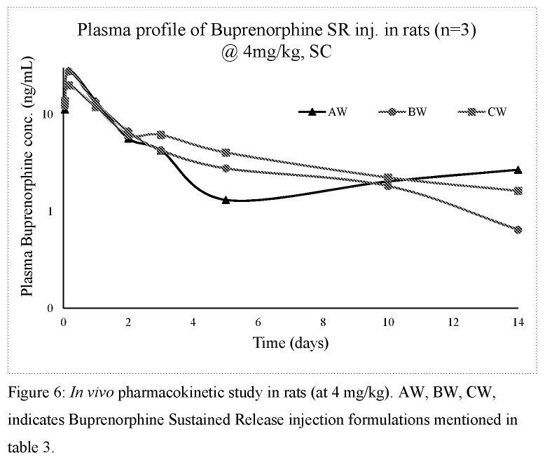

Male Sprague Dawley rats were administered formulations containing 4 mg/kg buprenorphine using #23 gauge needle, subcutaneously. Blood samples were withdrawn up to 14 days & plasma concentration was determined using LCMS. The results (FIG. 6) showed that a formulation of buprenorphine prepared with phospholipids resulted into controlled delivery of buprenorphine in vivo which is evident from more uniform plasma concentration of buprenorphine in the composition with phospholipid.

We claim:

1. A sustained release injectable pharmaceutical composition, comprising:
a. buprenorphine, its metabolite, a prodrug, or a salt thereof present in the concentration of about 0.5% w/w to 40.0% w/w of total composition;
b. a vehicle at a concentration of about 5% w/w to 95% w/w of total composition;
c. a release retarding agent at a concentration of about 0.2% w/w to 40% w/w of total composition; and
d. a stabilizer at a concentration of about 5% w/w to 70% w/w of total composition.

2. The sustained release injectable pharmaceutical composition of claim 1, wherein the vehicle is selected from the group consisting of the following or a mixture thereof:
a) a non-aqueous solvent selected from, polyethylene glycol, propylene glycol, benzyl benzoate, benzyl alcohol, isopropyl myristate, ethyl oleate, fatty acid esters and medium chain triglycerides and
b) a lipophilic solvent selected from soybean oil, sesame oil, peanut oil, cottonseed oil, castor oil and arachis oil.

3. The sustained release injectable pharmaceutical composition of claim 1, wherein the release retarding agent is selected from the group consisting of the following or a mixture thereof:
a) a phospholipid selected from phosphocholines, phosphoglycerols, phosphoserines and sphingolipids and
b) a polyorthoester selected from polycaprolactone (PCL) or polylactic acid (PLA) and
c) a lipophilic agent selected from stearyl alcohol, cetostearyl alcohol, cetyl alcohol, stearic acid, palmitic acid, stearates, palmitates, waxes like carnauba wax, beeswax, and hydrogenated castor oil.

4. The sustained release injectable pharmaceutical composition of claim 3, wherein the phosphocholine is selected from the group consisting of hydrogenated soya phosphatidyl choline (HSPC), 1,2-distearoyl-sn-glycero-3-phosphocholine (DSPC), dipalmitoylphosphatidylcholine (DPPC), 1,2-dimyristoyl-sn-glycero-3-phospho-choline (DMPC), 1-palmitoyl-2-oleoyl-sn-glycero-3-phosphocholine (POPC), soya phosphatidyl choline (SPC) and soya-lecithin.

5. The sustained release injectable pharmaceutical composition of claim 1, wherein the composition further includes an additive.

6. The sustained release injectable pharmaceutical composition of claim 5, wherein the additive is selected from the group consisting of cholesterol, Ethyl Hexyl Oleate, glyceryl esters like glyceryl monooleate, glyceryl monostearate, glyceryl palmitate, glyceryl palmitostearate, glyceryl dibehenate, glyceryl distearate, glyceryl isostearate, glyceryl laurate, glyceryl mono and dicaprylocaprate, glyceryl mono and dipalmitostearate, glyceryl monocaprylate, glyceryl monocaprylocaprate, glyceryl monocitrate, glyceryl oleate, glyceryl palmitate, glyceryl ricinoleate, glyceryl stearate, glyceryl stearate/peg-100 stearate, glyceryl tristearate, polyoxyl glyceryl stearate, sucrose esters, glycol esters.

7. The sustained release injectable pharmaceutical composition of claim 1, wherein the stabilizer is selected from the group consisting of benzyl alcohol, butylated hydroxyl anisole (BHA), butylated hydroxyl toluene (BHT) and a-tocopherol.

8. The sustained release injectable pharmaceutical composition of claim 1, wherein the composition is administered by subcutaneous injection or intramuscular injection.

9. The sustained release injectable pharmaceutical composition of claim 1, wherein the pharmaceutical composition after single administration will deliver buprenorphine over a period of about one week to 12 weeks, thereby reducing daily dose administration of buprenorphine.

10. Stable formulation in a prefilled syringe, with the fill volume ranging from 0.1 ml to 4 ml, delivering a single dose of the said formulation in claim 1.

11. A method of treating opioid addiction, acute pain and chronic pain, comprising administering an effective amount of Buprenorphine from the composition of claim 1.
